(19) United States
(12) Patent Application Publication
(10) Pub. No.: US 2011/0312016 A1
(43) Pub. Date: Dec. 22, 2011

(54) Method for Evaluating Immunosuppression

(75) Inventors: Oleh G. Pankewycz, Amherst, NY (US); Mark R. Laftavi, Buffalo, NY (US); Hans Minderman, Lockport, NY (US)

(73) Assignees: Health Research Inc., Buffalo, NY (US); The Research Foundation of State University of New York, Amherst, NY (US)

(21) Appl. No.: 13/128,292

(22) PCT Filed: Nov. 11, 2009
(86) PCT No.: PCT/US09/64010

§ 371 Date: Sep. 1, 2011

Related U.S. Application Data
(60) Provisional application No. 61/113,381, filed on Nov. 11, 2008.

(51) Int. Cl. C12Q 1/02

(52) U.S. Cl. 435/29

(57) ABSTRACT

Provided is a method for determining immunosuppression in an individual. The method entails obtaining a sample of blood from an individual, contacting cells in the blood sample with an activating agent to obtain activated cells, measuring the amount of nuclear NFkB in the activated cells, and comparing the amount of nuclear NFkB in the activated cells to a control. An amount of nuclear NFkB that is higher than the control is considered to be indicative of insufficient immunosuppression in the individual. An amount of nuclear NFkB that is lower than the control is considered to he indicative of excessive immunosuppression in the individual. An amount of nuclear NFkB that is the same as the control is considered to be indicative of an appropriate amount of immunosuppression in the individual.

This application claims priority to U.S. Provisional Patent Application No. 61/113,381, filed on Nov. 11, 2008, the disclosure of which is hereby incorporated by reference in its entirety.

### FIELD OF THE INVENTION

The present invention relates generally to determining immunosuppression, and more particularly to determining immunosuppression in an individual by measuring nuclear NFκB in blood cells obtained from the individual.

### BACKGROUND OF THE INVENTION

Patients who receive a solid organ transplant must take immunosuppressive therapy to prevent rejection. Contemporary immunosuppressive protocols call for continuous therapy for the life-span of the transplanted organ. Potentially life-long anti-rejection therapy has many adverse consequences including increased rates of infections and cancers, worsening cardiovascular risk factors and bone disease. Therefore, individualized or minimized immunosuppression is a major clinical goal, saving the highest levels of immunosuppressive therapy for those patients at highest risk of rejection and graft loss. Currently, there is an ongoing need for a reliable non-invasive test that allows for such patient directed immunosuppressive therapy. The present invention meets this need.

### SUMMARY OF THE INVENTION

The present invention provides a method for determining immunosuppression in an individual. The invention can be used to determine immunosuppression in any individual undergoing any type of immunosuppression with any immunosuppressive agent. Immunosuppression is considered to be a measure of immune competence.

The method comprises obtaining a sample of blood from an individual, contacting cells in the blood sample with an activating agent to obtain activated cells, measuring nuclear NFκB in the activated cells, and comparing the amount of nuclear NFκB in the activated cells to a control.

An amount of nuclear NFκB that is higher than the control is considered to be indicative of insufficient immunosuppression in the individual. An amount of nuclear NFκB that is lower than a control is considered to be indicative of excessive immunosuppression in the individual. An amount of nuclear NFκB that is the same as a control is considered to be indicative of an appropriate amount of immunosuppression in the individual.

In one embodiment, the method comprises obtaining a second whole blood sample from the individual (or dividing a first sample into first and second samples), wherein cells in the second sample are not activated. The amount of nuclear NFκB is the non-activated cells can be used for comparison to the amount of nuclear NFκB in the activated cells to establish a baseline amount of nuclear NFκB prior to activation.

The blood sample obtained from the individual comprises immune cells that include but are not limited to T cells, monocytes, polymorphonuclear leukocytes, eosinophils, and combinations thereof. In one embodiment, the cells analyzed in the method of the invention comprise CD3+ cells, CD4+ cells, CD8+, CD20+ cells, or a combination thereof. Thus, the blood sample contains lymphocytes that can be phenotyped according to particular surface antigens and analyzed in the method of the invention.

In one embodiment, the method further comprises communicating to a health care provider a determination that an amount of nuclear NFκB is indicative of insufficient, excessive or appropriate immunosuppression in the individual.

In one embodiment, the method further comprises modifying immunosuppression dosing for the individual subsequent to determining insufficient or excessive immunosuppression.

### BRIEF DESCRIPTION OF THE DRAWINGS

FIG. 1 provides a graphical representation of a comparison of imaging flow cytometry data with data obtained from a commercially available immunosuppression test. Discordant data were observed for 2/9 patients in which cases the patients suffered acute rejection (*) or viral infections (**), and thus demonstrates superiority of the present invention in predicting clinical outcomes.

FIG. 2 provides a graphical representation of a comparison of immune response of transplant recipients receiving immunosuppressive therapy (n=5) and healthy donors (n=4). Immune response was measured as the amount of nuclear NFκB translocation (similarity score) in CD3, CD4 and CD8 T-cells in response to ex-vivo stimulation to PMA/ion or TNFα. Bars represent mean values with standard deviations.

### DETAILED DESCRIPTION OF THE INVENTION

The present invention provides a method for determining immunosuppression in an individual. The method is based on our discovery that quantification of nuclear NFκB in activated immune cells obtained from an individual is indicative of the state of immunosuppression in the individual.

The method involves measuring in blood cells an amount of NFkB that translocates to the nucleus after the cells have been activated by exposure to an activating agent. Thus, in one embodiment, the invention entails determining a change in nuclear NFκB that occurs in response to activation of certain blood cells obtained from the individual. In an alternative embodiment, a ratio of cytoplasmic to nuclear NFκB can be determined in the activated cells.

“Immunosuppression” as used herein refers to the effect on the immune system of an individual elicited by administration of one or more immunosuppressive agents to the individual. Immunosuppression is considered a type of immune competence, meaning the degree to which an individual can mount an appropriate immune response against a foreign antigen. Immune competence of particular types of immune cells can also be determined in performance of the method of the invention.

In connection with immunosuppression, a wide variety of immunosuppressive agents are known in the art and are routinely administered to individuals for a variety of purposes. The invention is suitable for determining immunosuppression in any individual undergoing any type of immunosuppression with any immunosuppressive agent, which include but are not limited to calcineurin inhibitors (CNI) (i.e., tacrolimus or cyclosporin), mycophenolic acid (MPA) (i.e., Cellcept or Myfortic), sirolimus (i.e., Rapamune or Certican) and prednisone. Thus, it is considered that the method is suitable for evaluating the immunosuppression status of any mammal, including male and female humans, and ranging in age from infants to the elderly. In one embodiment, the invention is used to evaluate immunosuppression in an individual who has a transplanted organ or other tissue and is undergoing immunosuppression therapy to reduce the likelihood that the transplant will be rejected. In various embodiments, the individual has a transplanted kidney, pancreas, heart, lung, hand, face, skin, bone, bone marrow, cartilage, ligament or muscle. The transplant may also be a xenographic transplant, or a transplantation of a synthetic substance.

“Activation” or “activating” or to “activate” cells are terms understood to those skilled in the art. In general, activation comprises causing certain immune cells described more fully below to undergo an alteration in gene expression such that the cells can participate in effecting a more vigorous immune response relative to non-activated cells. Evidence of activation includes but is not limited to translocation of NFκB from the cytoplasm to the nucleus, as well as production of various cytokines by activated cells which promote, among other well known effects, an inflammatory response.

The method of the invention comprises obtaining a sample of blood from an individual, contacting cells in the blood sample with an activating agent to obtain activated cells, measuring nuclear NFκB in the activated cells, and comparing amount of nuclear NFκB in the activated cells to a control, suitable controls being more fully described below. The amount of NFκB that is translocated to the nucleus is considered to be a measure of NFκB translocation potential. It is considered that the NFκB translocation potential is indicative of the immune response that the cells would exhibit upon encountering a foreign antigen, such as an antigen displayed by transplanted tissue.

Determining more nuclear NFκB than in the control by performing the method of the invention is considered to be indicative of insufficient immunosuppression in the individual. In one embodiment, insufficient immunosuppression is exemplified by an individual who is undergoing immunosuppression therapy and experiences rejection of a transplanted organ or tissue. Those skilled in the art are familiar with clinical criteria used to determine whether any particular transplanted organ or tissue is being rejected. Less nuclear NFκB relative to the control is considered to be indicative of excessive immunosuppression in the individual. In one embodiment, excessive immunosuppression is exemplified by an individual who is undergoing immunosuppression therapy and experiences viral infections more frequently and/or with more severe symptoms than expected had the individual been receiving an appropriate amount of immunosuppression. Those skilled in the art are familiar with criteria used to determine whether any particular individual who is undergoing immunosuppression therapy is experiencing viral infections more frequently and/or with more severe symptoms than would be expected if an appropriate amount of immunosuppression was being provided to the individual. An amount of nuclear NFκB that is the same as a control is considered to be indicative of an appropriate amount of immunosuppression in the individual. One non-limiting example of an individual who has an appropriate amount of immunosuppression is an individual who has a transplanted organ or other transplanted tissue, is receiving immunosuppression, and is not rejecting the transplanted organ or other tissue, and is not experiencing viral infections.

The method of the invention is demonstrated to be superior to the commercially available assay marketed under the trade name ImmuKnow (Cylex, Columbia, Md.) assay in predicting clinical outcome for immunosuppressed individuals. In particular, our results demonstrate that in one individual, the ImmuKnow assay indicated heightened immunity yet the patient suffered from above normal viral infections. However, analysis of blood cells from that individual using an embodiment of the present invention showed a markedly diminished ability to translocate NFkB, which is consistent with excessive immunosuppression. In another individual, the ImmuKnow assay predicted excessive immunosuppression, but the patient had acute rejection. Analysis of blood cells from that individual using an embodiment of the present invention showed a greater than normal ability to translocate NFκB from cytoplasm to the nucleus, which is consistent with inadequate immunosuppression, and thus correctly reflected the actual clinical outcome for this patient.

As will be recognized from the foregoing, the present invention involves detecting amounts of nuclear NFκB in stimulated immune cells. NFκB is a ubiquitously expressed transcription factor that regulates many normal cellular processes. NFκB transcription proteins include a collection of proteins that exist as dimers of two classes of proteins. The Class A proteins, p105 and p100, do not ordinarily act as transcription factors unless they undergo limited proteolysis to the shorter proteins p50 and p52 respectively. Activated Class A proteins bind to Class B proteins c-Rel, RelB and p65 to form the activated heterodimeric transcription complex. The p65 subunit of NFκB is also referred to in the art as RelA, Rel A and RELA. The most avid dimer and the major NFκB complex is p50/RelA. The activation of NFκB is usually transient with nuclear localization lasting 30 to 60 minutes followed by rapid egress of NFκB back to the cytoplasm. Thus, the presence of nuclear NFκB is considered to represent a recent activation event.

It will be recognized by those skilled in the art that any subunit of which nuclear NFκB is comprised can be detected during performance of the method of the invention. For example, detecting any homo- or heterodimeric complexes containing NFκB p65 (RELA/p65), RELB, NFκB/p105, NFκB1/p50, REL and NFκB2/p52 can be performed to quantify nuclear NFκB. In one embodiment, nuclear NFκB is determined by detecting the p65 subunit of NFκB. In connection with this, any particular individual may have polymorphisms and/or other allelic variation in p65 (as well as other NFκB subunits), but it is considered that all such potential variations can be detected using commercially available reagents. For example, it is considered that p65 expressed in blood cells obtained from any individual human can be detected using any of a variety of commercially available anti-p65 antibodies, such as those available from ABCAM (Cambridge, Mass., USA) and a variety of other commercial vendors. In one embodiment, nuclear NFκB is determined by detecting human p65 protein that has the amino acid sequence designated by GenBank accession number CAA80524.2, Nov. 14, 2006 entry, which is incorporated herein by reference.

The blood sample obtained from the individual is one that comprises cells that are suitable for analysis using the method of the invention. Such cells include lymphocytes and other cells that participate in cell mediated and/or humoral immune responses. For example, cells that are present in the blood sample obtained from the individual and that can be analyzed in the method of the invention include but are not necessarily limited to T cells, monocytes, polymorphonuclear leukocytes, eosinophils, B cells, and combinations thereof. Those skilled in the art are familiar with known markers and methods that can be used to detect and differentiate these cell types from one another. For instance, T cells are CD3+ cells that can be further distinguished from each other by subtype markers, such as CD4+ (T helper cells) and CD8+ (cytotoxic T cells). Additionally CD19+ and/or CD20+ cells (B cells) and CD16+ cells (natural killer cells) can be analyzed in performing the method of the invention.

When cells in the sample of blood are activated, the activating agent is not particularly limited, and a wide variety of suitable activating agents are known in the art and are commercially available. Some non-limiting examples of activating agents suitable for use in the present invention include phytohaemagglutinin (PHA), phorbol 12-myristate 13-acetate (PMA) with ionomycin (ion), tumor necrosis factor alpha (TNF-alpha), and anti-CD3/CD28 antibodies.

Suitable controls for use in the method of the present invention include but are not limited to a standardized curve, cell lines with known proportions of cytoplasmic NFκB that translocates from the cytoplasm to the nucleus upon activation, or any other standardized parameter(s) that delineates a ratio of nuclear NFκB to cytoplasmic NFκB in blood cells after activation and that indicates appropriate, excessive or insufficient immunosuppression. Those skilled in the art will recognize how to interpret a comparison of the amount of nuclear NFκB to any particular control. For example, an amount of nuclear NFκB that is within the range of the amount of NFκB determined from lymphocytes obtained and activated from stable transplant patients (e.g., those patients not undergoing rejection or experiencing above normal viral infections) is considered to be the same as the control (e.g., the same as a normal control) and can be recognized as such by those skilled in the art. Likewise, an amount of nuclear NFκB that is above the range of the amount of nuclear NFκB for a normal control can be readily recognized, as can an amount of nuclear NFκB that is below a range of the amount of nuclear NFκB for the normal control.

In one embodiment, the blood sample obtained from the individual is divided into an experimental and a second blood sample. Cells in the experimental sample are contacted with the activating agent; cells in the second whole blood sample are not contacted with the activating agent (i.e., the second whole blood sample comprises non-activated cells). The amount of nuclear NFκB in the second whole blood sample can be determined according to the method of the invention and is considered to be a non-activated amount of nuclear NFκB. Thus, the non-activated cells can be used to establish a baseline, or non-activated, amount of nuclear NFκB for comparison with the amount of nuclear NFκB in the activated cells.

As described above, the activating agent used to activate the cells is not particularly limited. Those skilled in the art are also familiar with the incubation parameters used to activate any particular cell type(s) using any particular activating agent. In one embodiment, the activating agent is added to a blood sample and the cells and activating agent added thereto are incubated together for a period of from 1 minute to 60 minutes, including all integers there between. In one embodiment, the incubation period is 30 minutes. In one embodiment, the incubation period is not more than from 1 minute to 60 minutes, including all integers there between.

In one embodiment, the blood sample obtained from the individual is a sample of whole blood. It is an unexpected advantage of the present invention that cells in the whole blood, such as CD3+, CD4+, CD8+ cells, can be contacted with the activating agent in the sample of whole blood. This is considered to be a more accurate representation of the normal in vivo environment of the cells, relative to first separating the CD3+, CD4+, CD8+ cells out of whole blood (i.e., by using ficol gradients to isolate peripheral blood cells (PBL)) and then contacting the separated cells with the activating agent. Moreover, use of whole blood permits the assay to be completed in a much shorter amount of time than if separated immune cells are used. For example, the entire assay can be completed in not more than from 2 to 4 hours, as opposed to much longer periods for assays that rely on separated cells.

In one embodiment, after contacting the cells with the activating agent, cells are immunophenotyped with commercially available fluorescently labeled antibodies, after which the red blood cells are removed from the whole blood sample. Red blood cells can be removed using conventional techniques, such as by lysing using a hypotonic solution under conventional conditions which does not also result in lysis of the activated cells. In one embodiment, a commercially available lyse/fix solution (Becton Dickenson) can be used.

In one embodiment, prior to determining quantifying nuclear NFκB, the cells in which the nuclear NFkB is to be determined are incubated with an antibody specific to a relevant NFkB subunit conjugated to a fluorescent marker.

The relative amounts of nuclear NFκB can be determined in the activated and non-activated samples by analysis with commerically available devices and/or systems that can differentiate and quantify the nuclear and cytoplasmic NFκB, such as by a variety of digital microcopy-based imaging techniques. For example, activated and non-activated preparations of cells could be fixed and analyzed using detectably labeled antibodies to NFκB (such as to p65) and well known reagents to stain or otherwise identify the nucleus such that the nuclear (and if desired cytoplasmic) NFκB can be distinguished from one another. Suitable nuclear stains include but are not limited to 4′,6-diamidino-2-phenylindole (DAPI), Hoechst stains, Haematoxylin, Safranin, Carmine alum, and DRAQ5.

In one embodiment, nuclear NFκB can be determined using imaging flow cytometry. For example, the amount of nuclear NFκB in activated and non-activated cells can be determined for CD3+, CD4+, CD8+, and/or CD20+ cells using detectably labeled antibodies directed to the CD3+, CD4+, CD8+ and/or CD20+ molecules, as well as detectably labeled antibodies to NFκB. The nuclei of the cells can be simultaneously visualized using a suitable nuclear stain that can be detected by an imaging flow cytometer. In one embodiment, the nuclear stain is DAPI. In another embodiment, the nuclear stain is DRAQ5.

In one embodiment, the imaging flow cytometer is an image flow cytometer that is described in U.S. Pat. No. 7,522,758. The disclosure of U.S. Pat. No. 7,522,758 is hereby incorporated by reference.

In one embodiment, a relative amount of NFκB present in the nucleus can be represented by a similarity score determined using an imaging flow cytometer system such as that described in U.S. Pat. No. 7,522,758. In general, the smaller the similarity score, the less nuclear translocation of NFκB and vice versa. More specifically, the similarity score is considered to be a log transformed Pearson's Correlation coefficient of the pixel by pixel intensity correlation between the NFκB and nuclear stained (i.e., DRAQ5 image) which is calculated as a quantifiable parameter for the degree of NFκB translocation to the nucleus. The similarity score (+ or −) is determined from the slope of the regression line while it takes its value from how well the individual pixel data points fit the regression line (Pearson correlation). A very low degree of nuclear translocation yields a highly negative similarity score while a very high degree of nuclear translocation yields a highly positive similarity score. It will therefore be recognized that, in one embodiment a low degree of nuclear translocation can have anti-similar p65 and DRAQ5 images, while similar p65 and DRAQ5 images can yield a positive similarity score. Thus, in one embodiment, following stimulation, a negative similarity score obtained using an imaging flow cytometer system is indicative of excessive immune suppression, while a highly positive similarity score obtained using an imaging flow cytometer system is indicative of insufficient immune suppression. A standardized similarity score or ranges of similarity scores can accordingly be used as a control when performing the method of the invention.

Determining an amount of nuclear NFκB in activated cells from an individual, wherein the amount is different from a control, is considered to be indicative that the individual is a candidate for an alteration of his or her immunosuppression therapy. For example, an individual for whom performing the method of the invention indicates insufficient immunosuppression could be recommended for an increase in dosing, or for a change to a different immunosuppression agent. Likewise, an individual for whom performing the method of the invention indicates excessive immunosuppression could be recommended for a decrease in dosing, or for a change to a different immunosuppression agent. An individual for whom performing the method of the invention indicates an appropriate amount of immunosuppression could be recommended for no change in immunosuppression regime.

The method of the invention can be repeated to monitor the immunosuppression status of an individual over time. For example, the invention can be used to evaluate whether modifications of the immunosuppression therapy of an individual should be considered and/or implemented. The method of the invention can also be performed prior to initiation of immunosuppression therapy and compared to a sample(s) of blood obtained from the individual after initiation of immunosuppression therapy to evaluate the efficacy of the therapy.

In one embodiment, the method of the invention comprises communicating to a health care provider the result of determining an amount of nuclear NFκB in activated cells from an individual that is different from, or the same as, a control. The health care provider can be any individual who participates in making health care decisions for the individual. In another embodiment, the invention comprises communicating to an insurance provider the result of determining an amount of nuclear NFκB in activated cells from an individual that is different from, or the same as, a control.

In one embodiment, the method of the invention further comprises recommending an alteration of an immunosuppression therapy subsequent to determining an amount of nuclear NFκB that is different from a control, such as a control comprised of the amount of nuclear NFκB observed in activated cells obtained from stable transplant recipients. This embodiment may further comprise altering the immunosuppression therapy for the individual.

In one embodiment, the method comprises fixing the result of determining the amount of nuclear NFκB in a tangible medium of expression, such as a digital medium, including but not limited to a compact disk, DVD, or any other portable memory device. Thus, the invention also provides a device or other tangible medium that contains a machine or human readable result from determining nuclear NFκB that is different from that observed in a control.

The following Examples are intended to illustrate but not limit the invention.

### Example 1

We measured the degree to which NFκB translocation in peripheral T cells was impaired by immune-suppressive therapy using a commercially available imaging cell flow cytometer (Amnis Corporation, Seattle, Wash.).

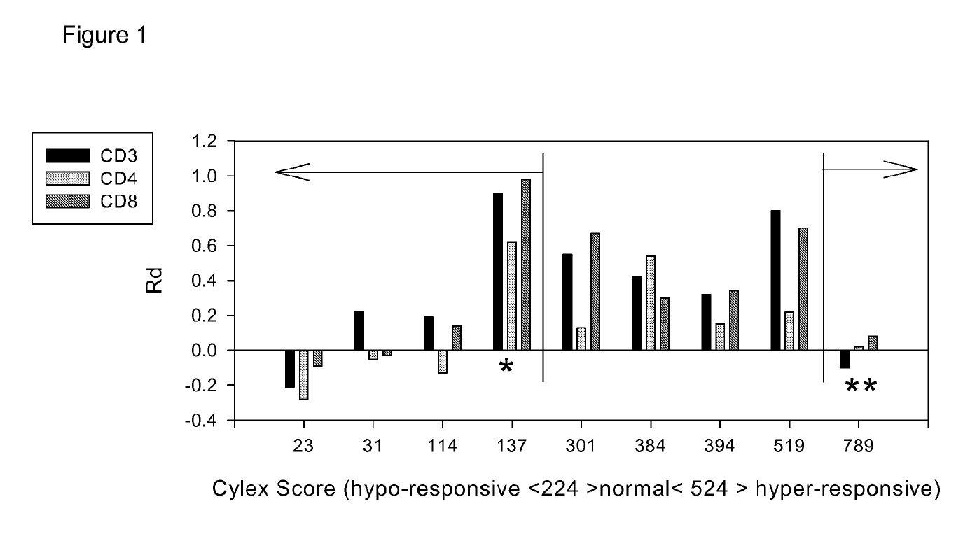

Peripheral blood cells from 9 transplant recipients were isolated, stimulated in culture with PMA/ionomycin (30 min), stained for T cell surface markers and NFκB (p65) and the relative amount of nuclear NFκB in resting and activated CD3, CD4 and CD8 positive T-cell subsets was compared. Results were then correlated with clinical response (stable graft function, infections and rejections) and ImmuKnow assay results. The assay correlated well with results obtained in parallel using the commercially available ImmuKnow product according to manufacturer's instructions, (FIG. 1) but there were 2 major discrepancies. In one patient (** in FIG. 1), the ImmuKnow assay levels indicated heightened immunity yet the patient suffered from major viral infections. In this patient, the imaging cell flow cytometry correctly showed a markedly diminished ability to translocate NFκB consistent with over-immunosuppression. In the other case (* in FIG. 1), the ImmuKnow assay levels predicted an excessive level of immunosuppression, but the patient had acute rejection. The imaging cell flow cytometry assay showed a greater than normal ability to translocate NFκB consistent with inadequate immunosuppression, thus correctly reflecting the clinical outcome. Thus, the present invention provides improved assessment of the degree of immunosuppression in an individual and is expected to more accurately predict clinical outcome across a broad range of patients.

### Example 2

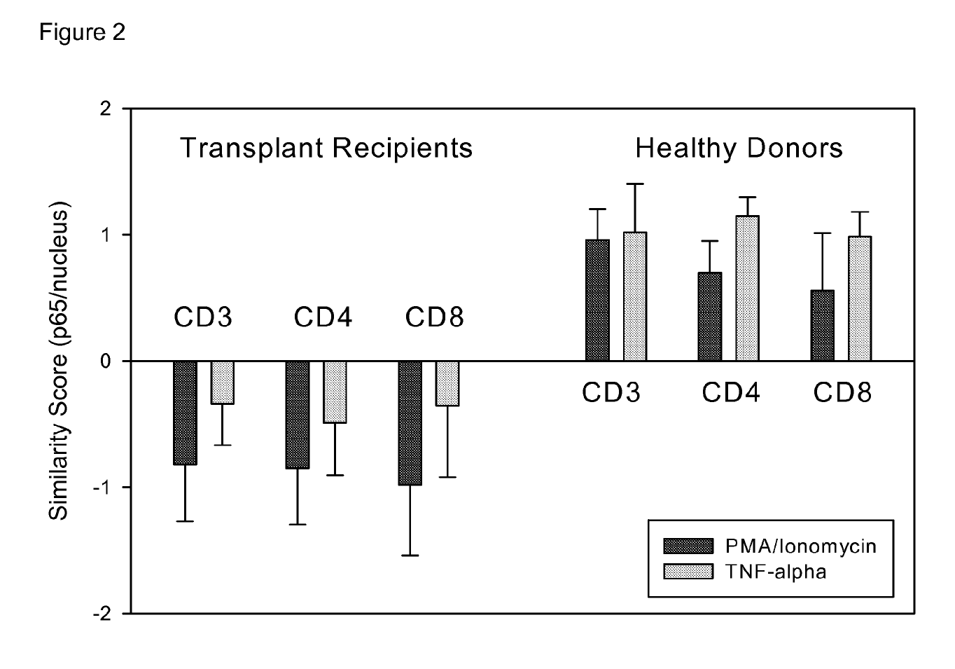

The assay described in Example 1 was modified to perform the stimulation and cell surface labeling in whole blood to enable the method to be performed in the normal environment of the cells and to permit faster performance of the assay. Using this approach, the immune response of 5 transplant patients as compared to 4 normal donors to stimulation to TNFα or PMA/ion was compared. The data depicted in FIG. 2 demonstrate that using a similarity score read-out for nuclear NFκB as a measure for immune response, a striking difference could be observed between the samples from normal donors and samples from transplant recipients undergoing immunosuppressive therapy. Thus, this Example unexpectedly demonstrates that the method of the invention is suitable analysis of the amount of nuclear NFkB using a procedure whereby immune cells are activated in whole blood.

While the invention has been described through illustrative examples, routine modifications will be apparent to those skilled in the art, which modifications are intended to be within the scope of the invention.

We claim:

1. A method for determining immunosuppression in an individual comprising:
i) obtaining a whole blood sample from the individual;
ii) contacting cells in the whole blood sample with an activating agent to obtain activated cells;
iii) determining an amount of nuclear NFκB in the activated cells; and
iv) comparing the amount of nuclear NFκB in the activated cells to a control;
wherein more nuclear NFkB relative to the control is indicative of insufficient immunosuppression in the individual;
wherein less nuclear NFkB relative to the control is indicative of excessive immunosuppression in the individual; and
wherein the same amount of nuclear NFkB as the control is indicative of an appropriate amount of immunosuppression in the individual.

2. The method of claim 1, wherein a second whole blood sample is obtained from the individual, wherein the second whole blood sample is not contacted with the activating agent, wherein a second amount of nuclear NFkB is determined from cells in the second whole blood sample to obtain a non-activated amount of nuclear NFkB, and wherein the non-activated amount of nuclear NFkB is compared to the amount of nuclear NFκB in the activated cells of claim 1.

3. The method of claim 1, wherein the activated cells are selected from the group consisting of T cells, B cells, monocytes, polymorphonuclear leukocytes, eosinophils, and combinations thereof.

4. The method of claim 1, wherein the activated cells are CD3+ cells, CD4+ cells, CD8+, CD20+ cells, or a combination thereof.

5. The method of claim 1, wherein the determining the amount of nuclear NFκB in the activated cells is performed using a detectably labeled antibody directed to a p65 subunit of the NFκB.

6. The method of claim 1, wherein the activating agent is selected from phorbol 12-myristate 13-acetate (PMA) with ionomycin (ion), and anti-CD3/CD28 antibodies.

7. The method of claim 1, wherein the individual is a recipient of an organ transplantation.

8. The method of claim 1, further comprising communicating to a health care provider a determination that the amount of nuclear NFkB is indicative of insufficient, excessive or appropriate immunosuppression in the individual.

9. The method of claim 1, further comprising modifying immunosuppression dosing for the individual subsequent to determining the amount of nuclear NFkB is indicative of insufficient or excessive immunosuppression.

10. The method of claim 1, wherein the individual is a human being.
